(19) United States
(12) Patent Application Publication
(10) Pub. No.: US 2026/0345873 A1
(43) Pub. Date: Nov. 5, 2026

(54) SYSTEMS, METHODS, AND DEVICES FOR POROUS PLASMA SPRAY ON ZIRCONIUM SUBSTRATE

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Vivek D. Pawar, Germantown, TN (US); Roger Ryan Dees, JR., Drummonds, TN (US); Nathaniel M. Lenz, Germantown, TN (US); Aline Elquist, Memphis, TN (US); Gordon B. Hunter, Germantown, TN (US); Amit Parikh, Memphis, TN (US); Zachary J. Post, Silver Spring, MD (US); Michael Dean Hughes, Cordova, TN (US); Paul C. Crabtree, JR., Nesbit, MS (US); Dawn McLean, Eads, TN (US); Sean M. Haddock, Germantown, TN (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 18/997,549

(22) PCT Filed: Aug. 3, 2023
(86) PCT No.: PCT/US23/29439

§ 371 Date: Jan. 22, 2025

Related U.S. Application Data
(60) Provisional application No. 63/370,408, filed on Aug. 4, 2022.
(60) Provisional application No. 63/370,410, filed on Aug. 4, 2022.

(51) Int. Cl. A61L 31/08; A61L 31/02; A61L 31/14

(52) U.S. Cl. CPC A61L 31/088; A61L 31/022; A61L 31/146; A61L 2420/02; A61L 2430/24

(57) ABSTRACT

A method for producing a bone ingrowth surface on a zirconium alloy device includes forming a substrate comprising a zirconium alloy, forming a substantially uniform oxide surface composition on an articulating surface of the substrate, and depositing a metallic or non-metallic coating on a bone apposition surface of the substrate. An interface between the bone apposition surface and the substrate has less than 20% surface residual oxide, less than 10% of surface or sub-surface hydrides, and less than 0.2% bulk hydrides.

### CROSS-REFERENCE TO RELATED APPLICATIONS

This is a non-provisional of, and claims the benefit of the filing date of, U.S. provisional patent application No. 63/370,408, filed Aug. 4, 2022, entitled “Porous Plasma Spray on Implants: Bone Ingrowth Surface on Zirconium Substrate,” and U.S. provisional patent application No. 63/370,410, filed Aug. 4, 2022, entitled “Porous Plasma Spray on Implants: Bone Ingrowth Surface on Zirconium Substrate, and Specifically, on Implants,” the entireties of which applications are incorporated by reference herein.

### FIELD OF THE DISCLOSURE

The present disclosure relates generally to surface modification of biocompatible materials, and more particularly to systems and methods of applying surface coatings to zirconium alloy devices.

### BACKGROUND OF THE DISCLOSURE

Zirconium and zirconium alloys possess several characteristics that make these materials useful for medical implant devices. These metal alloys have sufficient strength to withstand relatively high loads and, depending on the alloy components, are very corrosion resistant and biocompatible. In addition, the advent of oxidized zirconium has provided these materials with a surface that has superior hardness which is also resistant to brittle fracture, galling, fretting, and attack by bodily fluids.

In some particular applications, a Zr-2.5Nb alloy can be formed into a desired implant shape and then oxidized to produce a hard ceramic surface (e.g., for use in bearing against a polyethylene component). The ceramic surface is extremely abrasion resistant compared to traditional metal implant materials and also has a lower coefficient of friction against ultra-high molecular weight polyethylene (UHMWPE), the typical counter-face material used in total joint replacements. This oxidized zirconium alloy material can further provide these beneficial surface properties of a ceramic while retaining the beneficial bulk properties of the underlying metal (e.g., manufacturability, fracture toughness, and ductility), providing a good solution for these medical implant applications.

It can further be beneficial to add a metallic or non-metallic porous coating to the oxidized zirconium substrate or device to provide initial fixation of the implant immediately after surgery as well as to facilitate long-term stability by enabling bone growth onto and into the porous coating structure. Various approaches to attach bone ingrowth surfaces to oxidized zirconium compositions such as Zr-2.5Nb have been attempted. Such approaches have included texturing the surface using chemical or electrochemical means, plasma sprayed porous coating for medical implants, bonding of porous structure using hot isostatic process, bonding of porous structures using pulsed current sintering, and bonding of porous structure using heat and pressure.

There are challenges created, however, by high temperature application of coatings. For example, hydrogen is often used as a component of the feed gas during thermal spraying processes to produce certain desirable coating attributes, and the titanium powder used for thermal spraying contains hydrogen as it is produced using hydride-dehydride route. Zirconium is prone to hydrogen absorption, however, especially at elevated temperatures. As a result, this hydrogen can diffuse into the zirconium substrate, forming brittle zirconium hydride precipitates that can act as crack nucleation sites or propagation pathways when the material is put under load, thus making it more susceptible to static or fatigue fracture. Approaches to mitigate hydride issues include conducting a heat treatment to dissolve some or all hydrides back in Zr-2.5Nb substrate, asking suppliers to remove hydrogen gas from their feed gas (and use only helium and argon instead), placing specification limits in place on hydrogen content in Zr-2.5Nb bar-stock and forgings, and using a zirconium alloy that is less susceptible to hydride formation. Alloys that can resist hydride formation may contain tin, however, and thus may not be biocompatible.

In addition, thermal processes such as sintering can also negatively affect the mechanical properties of oxidized zirconium alloys, which may limit the types of coatings that can be applied to an oxidized zirconium substrate.

As a result, it would be desirable for systems and methods to add a metallic or non-metallic porous coating to an oxidized zirconium substrate without detrimentally impacting the substrate's mechanical properties and biocompatible properties.

### SUMMARY OF THE DISCLOSURE

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

A method for producing a bone ingrowth surface on a zirconium alloy device is disclosed. The method includes forming a substrate comprising a zirconium alloy; forming a substantially uniform oxide surface composition on an articulating surface of the substrate; and depositing a metallic or non-metallic coating on a bone apposition surface of the substrate; wherein an interface between the bone apposition surface and the substrate has less than 20% surface residual oxide, less than 10% of surface or sub-surface hydrides, and a total hydride volume fraction of a bulk of the substrate is less than 0.2%. In any preceding or subsequent example, the zirconium alloy can comprise Zr-2.5Nb.

In any preceding or subsequent example, oxide can be removed from the bone apposition surface prior to depositing the metallic or non-metallic coating.

In any preceding or subsequent example, depositing the metallic or non-metallic coating can comprise applying a sprayed or vaporized coating selected from the group consisting of a porous plasma spray, a laser engineered net shaping (LENS) system, a directed energy deposition, a cold spray, or a physical or chemical vapor deposition.

In any preceding or subsequent example, depositing the metallic or non-metallic coating can comprise spraying powders comprising titanium, zirconium, or alloys thereof on the bone apposition surface.

In any preceding or subsequent example, depositing the metallic or non-metallic coating can comprise introducing oxygen to form in-situ oxide at the bone apposition surface.

In any preceding or subsequent example, depositing the metallic or non-metallic coating can include applying a barrier layer on the bone apposition surface and depositing the metallic or non-metallic coating over the barrier layer, where the barrier layer can comprise a hydrogen composition that is lower than a hydrogen composition of the metallic or non-metallic coating.

In any preceding or subsequent example, depositing the metallic or non-metallic coating can comprise forming a surface composition having a hydrogen content at the bone apposition surface characterized by a surface composition that is greater than a baseline concentration in the substrate but drops asymptotically from the surface composition to the baseline concentration at a depth of less than 20 microns.

In any preceding or subsequent example, depositing the metallic or non-metallic coating can comprise forming a surface composition in which hydrides formed have lengths between about 5 microns and about 200 microns.

In any preceding or subsequent example, depositing the metallic or non-metallic coating comprises applying an anodic electric potential to the substrate.

In any preceding or subsequent example, the method can further include post processing the bone apposition surface to modify a pore morphology of the bone apposition surface. In some examples, post processing the bone apposition surface comprises solidifying loose particulates. In some examples, post processing the bone apposition surface comprises removing loose particulates. In some examples, post processing the bone apposition surface comprises applying a heat treatment configured to dissolve hydrides in the bone apposition surface.

In any preceding or subsequent example, the method can further include post processing the articulating surface to modify a surface roughness of the articulating surface to have an average surface roughness (Ra) less than 0.1 micron.

A zirconium alloy prosthesis is also disclosed. In some examples, the zirconium alloy prosthesis can include a substrate comprising a zirconium alloy; an articulating surface formed on the substrate, the articulating surface comprising oxide and an underlying diffusion hardened zone; and a bone apposition surface formed on the substrate, the bone apposition surface comprising a metallic or non-metallic coating formed on the substrate; wherein an interface between the bone apposition surface and the substrate has less than 20% surface residual oxide, less than 10% of surface or sub-surface hydrides (e.g., in some examples less than 3%), and less than 0.2% bulk hydrides. In some examples, the oxide of the bearing surface can range from 1 to 20 micron and diffusion hardened zone can range from 1 to 50 micron.

In any preceding or subsequent example, the zirconium alloy can comprise Zr-2.5Nb.

In any preceding or subsequent example, the bone apposition surface can comprise a porous titanium surface composition.

In any preceding or subsequent example, the interface between the bone apposition surface and the substrate has a hydrogen content at the bone apposition surface characterized by a surface composition that is greater than a baseline concentration in the substrate but drops asymptotically from the surface composition to the baseline concentration at a depth of less than 20 microns.

In any preceding or subsequent example, the interface between the bone apposition surface and the substrate has a composition in which hydrides formed have lengths between about 5 microns and about 200 microns.

Examples of the present disclosure provide numerous advantages. For example, in accordance with one or more features of the present disclosure, a metallic or non-metallic coating can be deposited on a bone apposition surface of a zirconium substrate while limiting the development of zirconium hydride precipitates and also preserving the mechanical properties of the oxidized zirconium alloy. In addition, the present methods can be performed in a controllable manner such that the ingrowth surface has desirable characteristics with respect to the attachment strength of the coating, the coating thickness, and the pore morphology.

Further features and advantages of at least some of the examples of the present disclosure, as well as the structure and operation of various examples of the present disclosure, are described in detail below with reference to the accompanying drawings.

### BRIEF DESCRIPTION OF THE DRAWINGS

By way of example, specific examples of the disclosed device will now be described, with reference to the accompanying drawings, in which:

FIG. 1 is a flow chart that illustrates a method for producing a bone ingrowth surface on a zirconium alloy device in accordance with one or more features of the present disclosure;

FIG. 2 is a side view of a zirconium alloy device in accordance with one or more features of the present disclosure;

FIGS. 3A and 3B illustrate top views of fixation surfaces of a zirconium substrate before a coating is applied in accordance with one or more features of the present disclosure;

FIGS. 4A and 4B illustrate side sectional views of fixation surfaces to which a coating is applied in accordance with one or more features of the present disclosure;

The drawings are not necessarily to scale. The drawings are merely representations, not intended to portray specific parameters of the disclosure. The drawings are intended to depict various examples of the disclosure, and therefore are not considered as limiting in scope. In the drawings, like numbering represents like elements.

### DETAILED DESCRIPTION

Systems, methods, and devices for producing a bone ingrowth surface on a zirconium alloy device are described more fully herein with reference to the accompanying drawings, in which one or more features of the zirconium alloy device will be shown and described. It should be appreciated that the various features may be used independently of, or in combination, with each other. It will be appreciated that the zirconium alloy device as disclosed herein may be embodied in many different forms and may selectively include one or more concepts, features, or functions described herein. As such, the zirconium alloy device should not be construed as being limited to the specific examples set forth herein. Rather, these examples are provided so that this disclosure will convey certain features of the zirconium alloy device to those skilled in the art.

As discussed above, it would be beneficial to add a metallic or non-metallic porous coating to an oxidized zirconium substrate or device, such as a Zr-2.5Nb substrate, for the purposes of bone ingrowth, press-fit, or for other applications. Metallic coatings that can be applied to an oxidized zirconium substrate without significant thermal effects can include a sprayed or vaporized coating, such as by using a porous plasma spray, a laser engineered net shaping (LENS) system, a directed energy deposition, a cold spray, and/or a physical or chemical vapor deposition. Although various examples described below make particular reference to porous plasma spraying, those having ordinary skill in the art will appreciate that any of a variety of other methods for applying a porous coating can be used in which energy is applied to melt or evaporate particles and deposit them on the substrate.

When applying a sprayed coating to an oxidized zirconium substrate, however, particularly one sprayed at a relatively high temperature, microstructure changes and hydride formation in the substrate may occur. The hydride formation could occur at temperatures as low as 100 °C, and typical temperature of the substrate may momentarily rise to 500 °C or more depending on the process conditions. Microstructure changes may inhibit the formation of a uniform, high-integrity oxide and reduce mechanical properties of the component. Hydrides in a large enough quantity are known to embrittle the alloy and can be detrimental to coating attachment strength when present at the interface between the coating and the substrate. Hydrides of great enough size and number can weaken the oxidized zirconium substrate. It is desired to reduce the total amount of hydrides that form and remain in the oxidized zirconium substrate as a result of the spraying process in order to maintain the oxidized zirconium substrate's mechanical properties.

Additionally, there are challenges related to adequately controlling attachment strength of the coating, coating thickness, and pore morphology. Attachment strength should be sufficient to avoid delamination of the bone ingrowth surface in-vivo. The coating thickness can vary depending on the desired press-fit. This in turn depends on the instrumentation and the anatomical design to be engaged (e.g., a knee femoral component or an acetabular cup). In any configuration, the bone ingrowth surface created by the coating application should have sufficiently large pore size and porosity to allow adequate bone growth and long-term fixation without loosening of the implant. In addition, the issue of microstructure is particularly relevant to zirconium alloys. Formation of undesirable phases such as hydrides may impact the fatigue strength and coating adhesion.

To address these issues, in accordance with one or more features of the present disclosure, the application of a metallic or non-metallic ingrowth coating deposited on the bone apposition surface of a zirconium alloy implant device using a plasma spray deposition process is disclosed. As will be described in greater detail herein, in some examples, titanium is used as the coating material, although the present methods can include spraying zirconium or zirconium alloy powder instead of titanium, or lower melting point alloy powders, such as TiH₂ or TiCo eutectic, can be added to the mixture of powder being sprayed. Regardless of the powder composition used, the coating deposition process can be designed to control the coating thickness, pore morphology, and/or attachment strength, all while minimizing negative effects on the substrate to reduce hydrides and maintain mechanical properties.

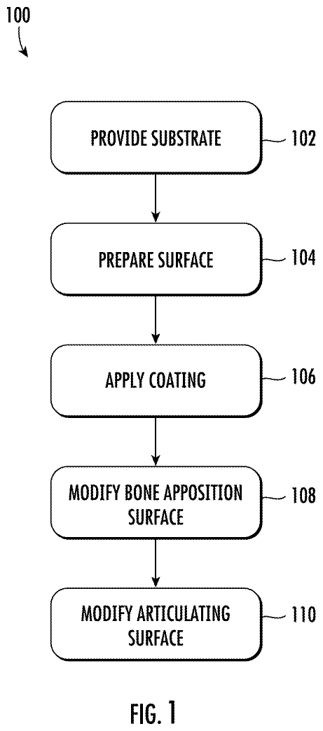

Referring to FIG. 1, in one aspect, the present subject matter provides a method 100 for producing a bone ingrowth surface on a zirconium alloy device. In this regard, in some examples, a substrate provision step 102 can include forming or otherwise providing a substrate comprising a zirconium alloy, the substrate having a desired implant shape. Such a substrate can be formed using high temperature forging, casting or machining from a bar stock, and/or powder metallurgy methods such as additive manufacturing and metal injection molding.

In some examples, a surface preparation step 104 can further be performed, where the substrate is oxidized to provide a desirable articulating surface for use in bearing against the counter-face material using in joint implants, to prepare the substrate to receive one or more coating layers, or to otherwise prepare one or more surface of the substrate for further processing. As discussed above, in some examples, the surface preparation step 104 can include oxidizing one or more surface of the substrate to form a ceramic material layer. In this regard, in some examples, the surface preparation step 104 can include a substantially uniform oxide surface being formed at least on an articulating surface of the substrate. Those having ordinary skill in the art will recognize, however, that although examples discussed herein refer to oxidation of zirconium to form a ceramic surface, improved medical implant devices can be achieved in which the surface of the zirconium substrate is modified to include oxides, carbides, nitrides, borides, and any combination thereof.

Alternatively, or in addition, in some examples, the surface preparation step 104 can further include modifying the substrate's microstructure to prepare it to receive a coating of other material layers, such as by heating just the surface to increase grain size and limit hydrogen diffusion along the grain boundaries.

In some examples, the surface preparation step 104 can include removing at least partially any oxide on the bone apposition surface on which bone ingrowth coating will be deposited. Removal of at least a portion of the oxide before plasma spray can enhance adhesion strength of the coating. Such removal of oxide can be accomplished by mechanical means. Such mechanical means can include blasting of the surface with ceramic grit or machining or milling. In addition, removing only a portion of the oxide can prevent formation of hydrides underneath the surface, where oxide is present, since the oxide may act as a barrier to hydrogen diffusion therefore, reducing the total amount of hydrides formed. The selective removal of oxide at the bone apposition surface can thus be controlled to provide a desired balance between removal of a sufficient amount of oxide to improve coating adhesion while maintaining some oxide as a barrier to help inhibit hydride diffusion.

In some examples, the surface preparation step 104 can include texturing the surface before plasma spray to allow undercuts that will further enhance adhesion of the coating. Texturing can be accomplished by mechanical or chemical means. In some examples mechanical methods including milling, turning, grit-blasting, or shot-blasting can be used, such as to chip away some of the surface and deform it into relative peaks and valleys. Alternatively, or in addition, in some examples, chemical methods can be used to texture the surface, such as by selectively etching the surface with chemicals such as acids or by anodic dissolution. Regardless of the methods used, the surface texturing can be performed in a manner selected to balance the advantage of a rougher surface that provides more adhesion against issues created by more dimensional surface variations that could reduce the effectiveness of fitting on the cut bone surfaces.

With the substrate being prepared to receive further surface materials, the method 100 can further include applying an ingrowth coating using a plasma spraying process 106. As discussed above, in some examples, the ingrowth coating can include titanium, zirconium, a zirconium alloy, lower melting point alloy powders, such as TiH₂ or TiCo eutectic, or combinations thereof. The plasma spraying process 106 can involve a thermal spraying coating method in which plasma is generated within a flowing gas stream to which a powder material is added. In some examples, the gas flows may range from 20-100 l/min, and the ratio of argon to helium in the gas mixture can range from 0.4 to 0.8. In some examples, the chamber pressure may range from 100-1000 mbar.

In some examples, hydride content can be reduced by reducing the hydrogen content of the materials used in the spraying process 106. For instance, in some examples, the material used for the zirconium substrate can be selected to itself have limited hydrogen content. As an example, hydrogen content of the substrate can be less than 12 parts per million. Alternatively, or in addition, other process materials can be selected to reduce their contribution to the hydrogen content in the finished product. In some examples, the hydrogen gas and water vapor content of the feed gas can be reduced, such as by heating the powder before spraying and/or by holding the powder in a vacuum before spraying to drive off moisture. In some examples, a small amount of oxygen can be added in the thermal plasma spray gas to generate in-situ oxide formation and thus reduce hydrogen pick-up. The standard free energy of formation of ZrO₂ (monoclinic) at 25 °C (298.15 K) is approximately 1042.746 kJ mol-1. In comparison, it is 64.686 kJ mol-1 for crystalline zirconium hydride (ZrH) and 124.281 kJ mol-1 for epsilon-ZrH₂. Although these values are for room temperature and for standard states of the materials, they do point to the fact that thermodynamically zirconium oxide is more likely to form than zirconium hydride in presence of same partial pressure of oxygen and hydrogen.

In some examples, the hydrogen and water content of the thermally sprayed powder can be reduced, such as through the selection of powders that contain significantly lower hydrogen than typically used for such processes. In some examples, the powder hydrogen content is approximately 200 ppm or less, with additional beneficial results being achieved using a powder having a hydrogen content between about 50 and 60 ppm or less. The use of low-hydrogen powder can reduce the maximum length and total number of large hydrides. In addition, in some examples, the spraying process 106 can include controlling the powder size and/or distribution to enable thinner coatings in certain areas.

Alternatively, or in addition, the spraying process 106 can include the application of multiple material layers having different compositions. In some examples, the spraying process 106 can include applying a barrier layer between the ingrowth coating and the substrate. In some examples, such a barrier layer can include low-hydrogen powder. Such a barrier layer can be 50-200 micron thick and can be denser than the porous surface formed near the bone apposition surface. In some examples, the spraying process 106 can include saturating the surface with an interstitial element such as oxygen, nitrogen, or carbon before plasma spray which could be accomplished before the plasma spray in the same chamber by using a different type of plasma. Alternatively, such treatment can be carried out separately. In some examples, such a saturation layer may range from 5 micron to 50 micron and can contain respective interstitial elements below the solubility limit. For example, zirconium can dissolve up to approximately 30 atomic percent oxygen before forming zirconium oxide, and thus it can be beneficial to keep oxygen concertation below 30 atomic percent.

In some examples, the spraying process 106 can include applying a dense coating near the substrate that is either made of titanium or titanium alloy. In some examples, this coating consists of an element that can act as a scavenger of hydrogen and thus reduced hydride formation, such as a vaporizable material that will scavenge hydrogen. Although not ideal for oxidized zirconium applications, in some examples, nickel can act as a scavenger in this regard.

In addition to selecting the composition and characteristics of the materials used to form the ingrowth coating, in some examples, the present methods can include controlling parameters of the plasma spray process 106 to reduce hydride formation. In some examples, controlling plasma spray parameters can include adjusting as nozzle distance from the substrate, pulse of the plasma, vacuum of the chamber, gas composition, plasma intensity, or other relevant process parameters that can be configured to control the characteristics of the ingrowth coating. Further, in some examples, the plasma spraying process 106 can include limiting the process temperature of the spraying process, increasing inert gas flow speed and/or volume, and/or increasing the wait times between sprayed layers. In some examples, the spray process 106 can include moving the plasma gun (without powder) on the plasma spray surface to solidify the surface particles. In this case, plasma characteristics can be identical to that used in spraying process but without the powder.

In addition to controlling process parameters, the spray process 106 can further be supplemented using additional tools or processes to control the application of the ingrowth coating. In some examples, the present methods can include using a sacrificial template, such as a carbon foam template, which is attached to the surface during the plasma spray, or the present methods can include applying a partial mask to control the thickness of the plasma spray. In addition, in some examples, the present method 100 can include compacting the powder using an external means during the plasma spray.

In addition to controlling the parameters of the plasma spray application, the spraying process 106 can further include controlling characteristics of the environment in which the spraying is performed. In some examples, the present methods can include keeping the temperature of the part being sprayed at or below approximately 160 °C to reduce diffusion of hydrogen in the part, which can result in a reduction in the maximum length and total number of large hydrides. Alternatively, or in addition, the present methods can include cooling the fixture holding the substrate. The cooling can be accomplished by a fixture that contains cavities for circulating chilled water or another fluid. Alternatively, cooling can be accomplished by switching the plasma off during the spray and allowing helium or argon gas to raster on the surface of the recently sprayed layer. In both cases, it can be advantageous to keep substrate temperature below 250 °C. Unlike some high temperature sintering processes, controlling the temperature of the present methods to be relatively low can help to retain the substrate microstructure and mechanical properties.

In addition, in some examples, the present methods can include applying a small electric potential (e.g., anodic) to the substrate during plasma spray to reduce the likelihood of hydrogen diffusion and thereby reduce hydride formation.

Alternatively, in some examples, rather than the entirety of the ingrowth coating being applied using a plasma spraying process, the present method 100 can include applying a pre-formed porous structure to the substrate and using a thermal spray to pin the base of the structure to the substrate. Such pre-formed structure can be made from traditional powder metallurgy processes or additive manufacturing. Such pre-formed structure may have slightly larger pore size and porosity to accommodate plasma spray material and changes to the structure during plasma spray or heating.

With the desired coatings applied, in some examples, the present method 100 can further include one or more post-processing steps that are configured to produce a desired surface composition and/or structure. In some examples, the present method 100 can optionally include one or more first post-processing step 108 configured to modify the bone apposition surface to have a desired composition and/or structure. In some examples, the one or more first post-processing step 108 can include mechanical manipulation, such as brushing, to remove loose particulates and create more open porosity on the surface for bone growth. In some examples, mechanical manipulation such as brushing is necessary to reduce particle release in-joint. In some examples, the one or more first post-processing step 108 can include heat treating after the plasma spray to dissolve the hydrides in the substrate and distribute hydrogen deeper in the substrate.

In some examples, the one or more first post-processing step 108 can include adding built-in landmarks on the femoral surface that enable thickness control during postprocessing.

In addition, in some examples, the present method 100 can optionally include one or more second post-processing step 110 configured to modify a surface roughness of the articulating surface. In some examples, the one or more second post-processing step 110 can include producing a surface finish having an average surface roughness (Ra) less than 0.1 micron. In some examples, the one or more second post-processing step 110 can be performed before one or both of the spraying process 106 or the one or more first post-processing step 108.

Variation in the order of these steps is possible. Additional steps may be added. Some steps may be removed. Multiple processes may be employed or only a single process step may be employed.

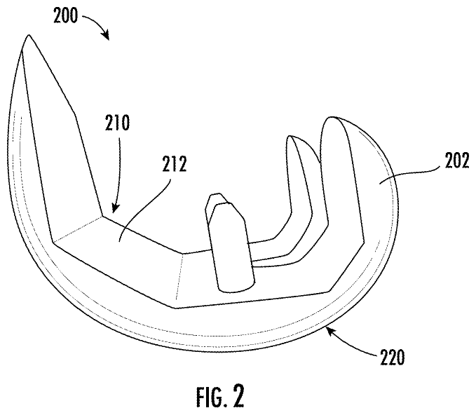

With reference to FIGS. 2-4B, examples of features of a zirconium alloy device 200 in accordance with one or more features of the present disclosure is shown. As illustrated, a medical device 200 can be made from a zirconium alloy substrate 202 with at least one surface coated with a metallic or non-metallic bone ingrowth coating. In some examples, the hydrogen content at the surface of the substrate immediately below the bone ingrowth coating is greater than that within the substrate but decreases in an asymptotic manner to the baseline substrate concentration (i.e., a total hydride volume fraction of a bulk of the substrate), such as at a depth of less than 20 microns. In some examples, the hydrogen content and hydrides formed decrease in an asymptotic manner at a depth of less than 200 microns. In some examples, the hydride length immediately below the bone ingrowth coating is between about 5 microns and about 200 microns. Smaller hydrides will reduce the risk of coating delamination during the use.

First, referring to FIG. 2, as discussed above, the medical device 200 (e.g., a knee femoral component) can include a substrate 202 comprising a zirconium alloy with a bone apposition surface 210 formed on the substrate 202. The bone apposition surface 210 includes thereon a metallic or non-metallic coating 212 formed on the substrate 202. The device 200 further includes an articulating surface 220 formed on the substrate 202, the articulating surface 220 comprising oxide and an underlying diffusion hardened zone.

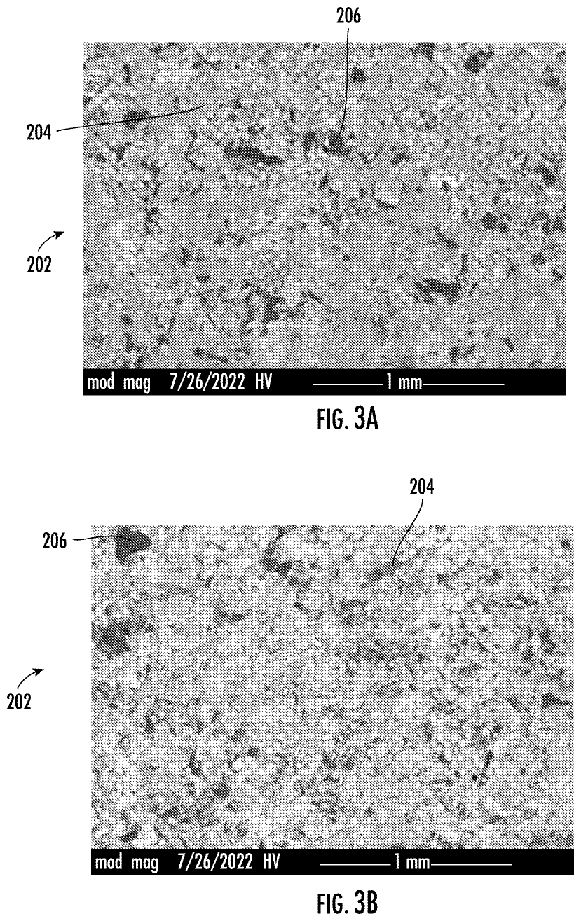

Referring to FIGS. 3A and 3B, as discussed above, surface oxides on the zirconium substrate 202 can be removed before the coating 212 is applied. In this regard, FIG. 3A illustrates an example of a zirconium substrate 202 on which insufficient oxide removal has been performed. In this example, approximately 35% residual oxide 204 remains (i.e., relative to 100% oxide representing an unmodified surface), along with other remnants of grit blast media 206. In other examples, as much as approximately 75% or more residual oxide can remain after incomplete removal. In contrast, in accordance with the methods discussed above, in some examples, residual oxide 204 at the surface of a zirconium substrate 202 can be removed prior to coating as shown in FIG. 3B. In this example, only approximately 16% residual oxide remains. In other examples, surface residual oxide levels of 20% or less can provide improved coating adhesion, with residual oxide values ranging from 5-18% providing particular benefit.

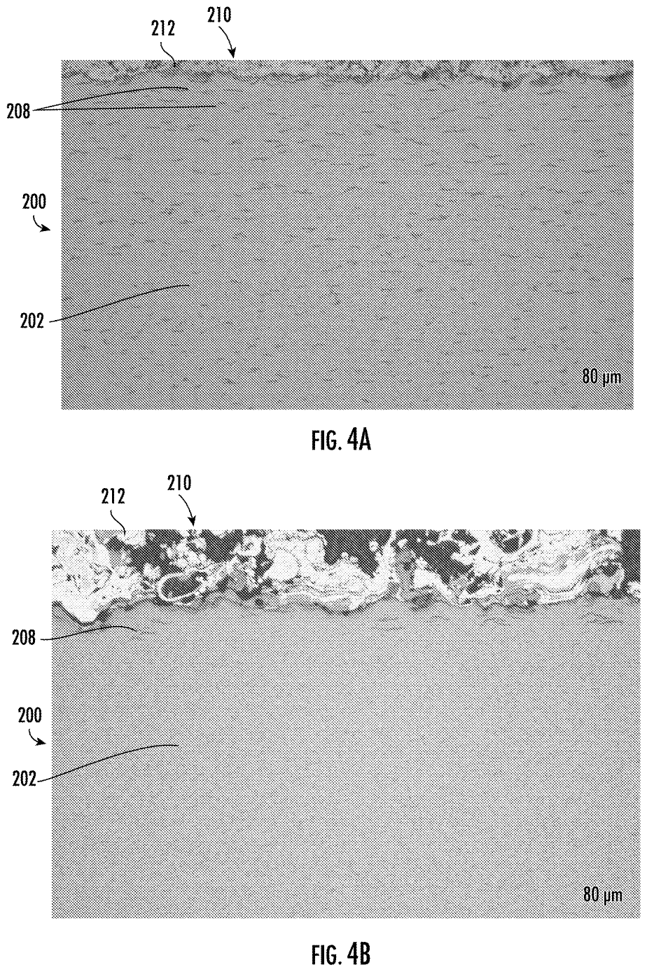

Further, referring to FIGS. 4A and 4B, as discussed above, it can be desirable to minimize the inclusion of sub-surface hydrides 208 at or near a surface of the zirconium substrate 202 on which a coating 212 is applied. Referring to FIG. 4A, an unacceptable level of hydride precipitation remains below the coating 212. In this example, the depth of precipitation is approximately 1000 microns. In some situations, unacceptable levels of sub-surface hydrides can result in coating delamination. By applying the methods discussed above, however, the formation of hydrides can be reduced. Referring to FIG. 4B, sub-surface hydrides are limited to less than 0.5%, although improvements in the microstructure can be observed with surface or sub-surface hydrides less than 10%.

While the present disclosure refers to certain examples, numerous modifications, alterations, and changes to the described examples are possible without departing from the sphere and scope of the present disclosure, as defined in the appended claim(s). Accordingly, it is intended that the present disclosure not be limited to the described examples, but that it has the full scope defined by the language of the following claims, and equivalents thereof. The discussion of any example is meant only to be explanatory and is not intended to suggest that the scope of the disclosure, including the claims, is limited to these examples. In other words, while illustrative examples of the disclosure have been described in detail herein, it is to be understood that the inventive concepts may be otherwise variously embodied and employed, and that the appended claims are intended to be construed to include such variations, except as limited by the prior art.

The foregoing discussion has been presented for purposes of illustration and description and is not intended to limit the disclosure to the form or forms disclosed herein. For example, various features of the disclosure are grouped together in one or more examples or configurations for the purpose of streamlining the disclosure. However, it should be understood that various features of the certain examples or configurations of the disclosure may be combined in alternate examples, or configurations. Any example or feature of any section, portion, or any other component shown or particularly described in relation to various examples of similar sections, portions, or components herein may be interchangeably applied to any other similar example or feature shown or described herein. Additionally, components with the same name may be the same or different, and one of ordinary skill in the art would understand each component could be modified in a similar fashion or substituted to perform the same function.

Moreover, the following claims are hereby incorporated into this Detailed Description by this reference, with each claim standing on its own as a separate example of the present disclosure.

As used herein, an element or step recited in the singular and proceeded with the word “a” or “an” should be understood as not excluding plural elements or steps, unless such exclusion is explicitly recited. Furthermore, references to “one example” of the present disclosure are not intended to be interpreted as excluding the existence of additional examples that also incorporate the recited features.

The phrases “at least one”, “one or more”, and “and/or”, as used herein, are open-ended expressions that are both conjunctive and disjunctive in operation. The terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure, and do not create limitations, particularly as to the position, orientation, or use of this disclosure. Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. All rotational references describe relative movement between the various elements. Identification references (e.g., primary, secondary, first, second, third, fourth, etc.) are not intended to connote importance or priority but are used to distinguish one feature from another. The drawings are for purposes of illustration only and the dimensions, positions, order and relative to sizes reflected in the drawings attached hereto may vary.

We claim:

1. A method for producing a bone ingrowth surface on a zirconium alloy device, the method comprising:
forming a substrate comprising a zirconium alloy;
forming a substantially uniform oxide surface composition on an articulating surface of the substrate; and
depositing a metallic or non-metallic coating on a bone apposition surface of the substrate;
wherein an interface between the bone apposition surface and the substrate has less than 20% surface residual oxide and less than 10% of surface or sub-surface hydrides.

2. The method of claim 1, wherein the zirconium alloy comprises Zr-2.5Nb.

3. The method of claim 1, wherein a total hydride volume fraction of a bulk of the substrate is less than 0.2%.

4. The method of claim 1, comprising removing oxide from the bone apposition surface prior to depositing the metallic or non-metallic coating.

5. The method of claim 1, wherein depositing the metallic or non-metallic coating comprises applying a sprayed or vaporized coating selected from the group consisting of a porous plasma spray, a laser engineered net shaping (LENS) system, a directed energy deposition, a cold spray, or a physical or chemical vapor deposition.

6. The method of claim 1, wherein depositing the metallic or non-metallic coating comprises spraying powders comprising titanium, zirconium, or alloys thereof on the bone apposition surface.

7. The method of claim 1, wherein depositing the metallic or non-metallic coating comprises introducing oxygen to form in-situ oxide at the bone apposition surface.

8. The method of claim 1, wherein depositing the metallic or non-metallic coating comprises:
applying a barrier layer on the bone apposition surface; and
depositing the metallic or non-metallic coating over the barrier layer;
wherein the barrier layer comprises a hydrogen composition that is lower than a hydrogen composition of the metallic or non-metallic coating.

9. The method of claim 1, wherein depositing the metallic or non-metallic coating comprises forming a surface composition having a hydrogen content at the bone apposition surface characterized by a surface composition that is greater than a baseline concentration in the substrate but drops asymptotically from the surface composition to the baseline concentration at a depth of less than 20 microns.

10. The method of claim 1, wherein depositing the metallic or non-metallic coating comprises forming a surface composition in which hydrides formed have lengths between about 5 microns and about 50 microns.

11. The method of claim 1, comprising post processing the bone apposition surface to modify a pore morphology of the bone apposition surface.

12. The method of claim 11, wherein post processing the bone apposition surface comprises solidifying loose particulates.

13. The method of claim 11, wherein post processing the bone apposition surface comprises removing loose particulates.

14. The method of claim 11, wherein post processing the bone apposition surface comprises applying a heat treatment configured to dissolve hydrides in the bone apposition surface.

15. The method of claim 1, comprising post processing the articulating surface to modify a surface roughness of the articulating surface to have an average surface roughness (Ra) less than 0.1 micron.

16. A zirconium alloy prosthesis comprising:
a substrate comprising a zirconium alloy;
an articulating surface formed on the substrate, the articulating surface comprising oxide and an underlying diffusion hardened zone; and
a bone apposition surface formed on the substrate, the bone apposition surface comprising a metallic or non-metallic coating formed on the substrate;
wherein an interface between the bone apposition surface and the substrate has less than 20% surface residual oxide, less than 10% of surface or sub-surface hydrides, and a total hydride volume fraction of a bulk of the substrate is less than 0.2%.

17. The prosthesis of claim 16, wherein the zirconium alloy comprises Zr-2.5Nb.

18. The prosthesis of claim 16, wherein the bone apposition surface comprises a porous titanium surface composition.

19. The prosthesis of claim 16, wherein the interface between the bone apposition surface and the substrate has a hydrogen content at the bone apposition surface characterized by a surface composition that is greater than a baseline concentration in the substrate but drops asymptotically from the surface composition to the baseline concentration at a depth of less than 20 microns.

20. The prosthesis of claim 16, wherein the interface between the bone apposition surface and the substrate has a composition in which hydrides formed have lengths between about 5 microns and about 50 microns.
